(19) United States
(12) Patent Application Publication
(10) Pub. No.: US 2001/0044427 A1
(43) Pub. Date: Nov. 22, 2001

(54) Pharmaceutical kit

(76) Inventors: Sidney Mazel, Basking Ridge, NJ (US); James M. Mundt, Warrington, PA (US)

Correspondence Address: MERCK AND CO INC, P O BOX 2000, RAHWAY, NJ, 070650907

(21) Appl. No.: 09/737,329

(22) Filed: Dec. 15, 2000

Related U.S. Application Data
(60) Provisional application No. 60/246,891, filed on Nov. 9, 2000.
(60) Provisional application No. 60/186,983, filed on Mar. 6, 2000.
(60) Provisional application No. 60/172,750, filed on Dec. 20, 1999.

(51) Int. Cl. A61K 31/66; A01N 57/00; B65D 83/10

(52) U.S. Cl. 514/102; 206/363

(57) ABSTRACT

The present invention relates to pharmaceutical treatment kits and to methods for administering a pharmaceutical active.

### CROSS-REFERENCE TO RELATED APPLICATIONS

The present invention is related to U.S. provisional applications Ser. Nos. 60/246,891, filed Nov. 9, 2000, 60/186,983, filed Mar. 6, 2000, and 60/172,750, filed Dec. 20, 1999, the contents of which are hereby incorporated by reference.

### FIELD OF THE INVENTION

The present invention relates to pharmaceutical treatment kits and to methods for administering a pharmaceutical active. These kits are particularly useful for administering a pharmaceutical active according to a once weekly treatment regimen.

### BACKGROUND OF THE INVENTION

The present invention relates to pharmaceutical treatment kits and to methods for administering a pharmaceutical active. These kits are particularly useful for continuously administering a pharmaceutical active according to a once weekly treatment regimen.

Many forms of dispensing containers and other holders for pharmaceutical actives have been proposed and commercially introduced. Pharmaceutical agents, especially those in the form of tablets, pills, and capsules, are generally dispensed in vials, bottles, or blister packs. One deficiency with such packaging is that the user is responsible for maintaining an independent record by human memory or other means as to whether or not the proper dosage has actually been administered. This deficiency is particularly problematic for users who suffer from impaired memory performance or who may be taking multiple medications.

The lack of patient compliance with adhering to a drug administration program is a potential issue. Aside from the fact that the patient may not be receiving the intended therapeutic benefit from noncompliance, there are potentially more serious issues that can arise from noncompliance. Studies documenting patient noncompliance have shown rates ranging from about 15% to about 93%, depending upon the population studied and the medical regimen involved. See e.g., R. N. Greenberg, “Overview of Patient Compliance with Medication Dosing: A Literature Review”, Clinical Therapeutics, Vol. 6, No. 5, pp. 592-599 (1984), which is incorporated by reference herein in its entirety. Clearly there is a continuing unmet need to make patient compliance in the administration of a pharmaceutical active easier.

Many forms of dispensing kits, containers, and other holders for pharmaceutical actives have been proposed and commercially introduced. Many of these containers are intended for dispensing dosages of the pharmaceutical product on a daily basis. See e.g., U.S. Pat. No. 5,265,728, to Allendorf et al., issued Nov. 30, 1993; EP Publication 0 511 726 A2, to Berlex Laboratories, Inc., published Nov. 4, 1992; PCT Publication No. WO 99/51214, to Akzo Nobel, published Oct. 14, 1999; U.S. Pat. No. 3,677,397, to Beckham, issued Jul. 18, 1972; and U.S. Pat. No. 3,504,788, to Gray et al., issued April 7, 1970, which are all incorporated by reference herein in their entirety, and which describe dispensers for administering various pharmaceuticals, including oral contraceptives, on a continuous or daily basis. Other containers are designed for administering several dosages of the same medication per day or for concurrently or noncurrently administering two or more different pharmaceutical actives. See e.g., U.S. Pat. No. 4,736,849, to Leonard et al., issued Apr. 12, 1988; U.S. Pat. No. 4,295,567, to Knudsen, issued Oct. 21, 1981; U.S. Pat. No. 4,573,580, to Messer, issued Mar. 4, 1986; U.S. Pat. No. 4,889,238, to Batchelor, issued Dec. 26, 1989; and U.S. Pat. No. 5,927,500, to Godfrey et al., issued Jul. 27, 1999, which are all incorporated by reference herein in their entirely.

However, none of the aforementioned dispensers or kits are designed or intended for administering a pharmaceutical active according to a continuous schedule having a dosing interval of once weekly, i.e. for administering a unit dosage of pharmaceutical active once per week. Such a kit would be highly useful for administering a wide variety of active ingredients. For example, it has recently been disclosed that bisphosphonate actives such as alendronate can be administered according to a weekly dosing regimen in the treatment and prevention of disease states such as osteoporosis. See e.g., U.S. Pat. No. 5,994,329, to Daifotis et al., issued Nov. 30, 1999 and PCT Application No. WO 99/04773, to Daifotis et al., published Feb. 4, 1999, which are both incorporated by reference herein in their entirety. Also see S. Adami et al., “Rationale for Once Weekly Dosing Regimens with Alendronate for the Treatment and Prevention of Osteoporosis”, 6^{th} International Symposium on Clinical Disorders of Bone Metabolism, Venice, Italy, Abstract No. 48, Nov. 20, 1999; H. G. Bone, et al., “Alendronate 70 mg Once Weekly is Therapeutically Equivalent to Alendronate 10 mg Daily”, 6^{th} International Symposium on Clinical Disorders of Bone Metabolism, Venice, Italy, Abstract 55, Nov. 20, 1999; and T. Schnitzer et al., “Therapeutic equivalence of alendronate 70 mg once-weekly and alendronate 10 mg daily in the treatment of osteoporosis”, Aging Clin. Exp. Res., Vol. 12, No. 1, pp. 1-12, 2000, which are all incorporated by reference herein in their entirety. Even though such once weekly therapies represent a significant advance over conventional daily therapies, it would be highly desirable to foster the ease of administration and help ensure patient compliance with such once weekly therapies.

Clearly, the present invention thus represents a useful advancement over the prior art.

It is an object of the present invention to provide a kit for administering one or more units dosages of a pharmaceutical active according to a once weekly dosing regimen.

It is another object of the present invention to provide methods for administering a one or more unit dosages of a pharmaceutical active according to a once weekly dosing regimen.

It is another object of the present invention to provide such kits and methods for administering bisphosphonates.

It is another object of the present invention to provide such kits and methods for inhibiting bone resorption and for treating or preventing disease states involving bone resorption, such as osteoporosis.

These and other objects will become readily apparent from the detailed description which follows.

### SUMMARY OF THE INVENTION

The present invention relates to a kit for administering a pharmaceutical active according to a once weekly treatment regimen, said kit comprising:

(a) at least one unit dosage of a pharmaceutical active for administration according to a once weekly treatment regimen, and

(b) a card, said card further comprising:

(i) a least one printable surface,

(ii) a means for containing said unit dosage or dosages, and

(iii) a memory aid for administering said unit dosage or dosages.

The present invention also relates to kits comprising means for the weekly administration of a pharmaceutical active characterized in that the kit comprises:

(a) at least one unit dosage of a pharmaceutical active for administration according to a once weekly regimen, and

(b) a card, said card further comprising:

(i) at least one printable surface,

(ii) a means for containing said unit dosage or dosages, and

(iii) a memory aid for administering said unit dosage or dosages.

The present invention also relates to a method for administering a pharmaceutically active according to a continuous schedule having a dosing interval of once weekly comprising administering said pharmaceutical active from a kit comprising:

(a) at least one unit dosage of a pharmaceutical active for administration according to a once weekly regimen, and

(b) a card, said card further comprising:

(i) at least one printable surface,

(ii) a means for containing said unit dosage or dosages, and

(iii) a memory aid for administering said unit dosage or dosages.

The present invention also relates to a method for inhibiting bone resorption in a mammal in need thereof comprising orally administering to said mammal a pharmaceutically effective amount of a bisphosphonate as a unit dosage according to continuous schedule having a dosing interval of once weekly comprising administering said pharmaceutical active from a kit comprising:

(a) at least one unit dosage of a pharmaceutical active for administration according to a once weekly regimen, and

(b) a card, said card further comprising:

(i) at least one printable surface,

(ii) a means for containing said unit dosage or dosages, and

(iii) a memory aid for administering said unit dosage or dosages.

In one embodiment the means for containing said unit dosage or dosages is a blister.

In another embodiment the present invention relates to a kit wherein the card is a foldable card.

In another embodiment the present invention relates to a kit wherein the card is paperboard.

In another embodiment the present invention relates to a kit wherein the memory aid comprises a calendar for indicating the day of the week on which to administer said dosage or dosages.

In another embodiment the present invention relates to a kit wherein the memory aid comprises a removable sticker or stickers indicating the administration of said dosage or dosages.

In another embodiment the present invention relates to a kit wherein the card further comprises a slit or pocket.

In another embodiment the present invention relates to a kit which further comprises an optional second, removable unfolded card which can be contained in said slit or pocket.

In another embodiment the present invention relates to a kit wherein the memory aid comprises a removable sticker or stickers indicating the administration of said dosage or dosages and wherein said sticker or stickers are affixed to said removable unfolded optional second card.

In another embodiment of the present invention relates to a kit wherein the pharmaceutical active is a bisphosphonate.

In another embodiment of the present invention the unit dosages are oriented in the intended order of their use.

### BRIEF DESCRIPTION OF THE FIGURES

FIG. 1 is an exploded perspective view, on a reduced scale, of a pharmaceutical kit in the fully unfolded position with a main card and an uninserted insert card;

FIG. 2 is a full scale front plan view of the main card thereof;

FIG. 3 is a full scale end elevational view of the main card thereof;

FIG. 4 is a full scale rear plan view of the main card thereof;

FIG. 5 is a full scale right end elevational view of the main card thereof;

FIG. 6 is a full scale left end elevational view of the main card thereof;

FIG. 7 is a full scale front plan view of the insert card of the pharmaceutical kit shown in FIG. 1;

FIG. 8 is a full scale rear plan view of the insert card of the pharmaceutical kit shown in FIG. 1;

FIG. 9 is a full scale side elevational view of the insert card of the pharmaceutical kit shown in FIG. 1;

FIG. 10 is a full scale end elevational view of the insert card of the pharmaceutical kit shown in FIG. 1;

FIG. 11 is a full scale perspective view of the pharmaceutical kit shown in FIG. 1 in the fully folded position.;

FIG. 12 is an exploded perspective view, on a reduced scale of the pharmaceutical kit shown in FIG. 1, in the fully unfolded position with a partially inserted insert card;

FIG. 13 is a partial view of FIG. 2 showing the blister card being opened;

FIG. 14 is a partial rear view of FIG. 2 showing the blister card being opened;

FIG. 15 is a partial perspective view, on an enlarged scale, of FIG. 2 showing a pharmaceutical tablet being punched from the blister card;

FIG. 16 is a partial sectional view, on an enlarged scale, of FIG. 2 showing in detail the laminated card and blister feature; and

FIG. 17 is a full scale front plan view of the insert card of the pharmaceutical kit shown in FIG. 1 with the attached stickers partially removed.

### DETAILED DESCRIPTION OF THE INVENTION

The present invention relates to pharmaceutical treatment kits and to methods for administering a pharmaceutical active. These kits are particularly useful for continuously administering a pharmaceutical active according to a once weekly treatment regimen.

The term “inhibiting”, as used herein, is intended to include both treating and reducing the risk of contracting, i.e. preventing, the disease state or condition, such as, for example abnormal bone resorption or osteoporosis.

The term “therapeutically effective amount”, as used herein, means that amount of the bisphosphonate compound, that will elicit the desired therapeutic effect or response when administered in accordance with the desired treatment regimen. A preferred therapeutically effective amount of the bisphosphonate is a bone resorption inhibiting amount.

The term “pharmaceutically acceptable” as used herein means that the salts and esters and other derivatives of the bisphosphonates have the same general pharmacological properties as the free acid form from which they are derived and are acceptable from a toxicity viewpoint.

The term pharmaceutically acceptable salt or ester, as used herein refers to non-toxic salts or esters of the compounds useful in the instant invention which are generally prepared by reacting the free base when present with a suitable organic or inorganic acid or prepared by reacting one or more of the hydroxy groups of the phosphonate groups (when present) with an appropriate acylating agent. Representative salts include the following: acetate, benzenesulfonate, benzoate, bicarbonate, bisulfate, bitartrate, borate, bromide, calcium, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the-compounds of the invention carry an acidic moiety, suitable pharmaceutically acceptable salts thereof can include alkali metal salts, e.g., sodium or potassium salts; alkaline earth metal salts, e.g., calcium or magnesium salts; and salts formed with suitable organic ligands, e.g., quaternary ammonium salts. Pharmaceutically acceptable salts also specifically include hydrates as well as the anhydrous forms. Pharmaceutically acceptable esters include acetates, propionates, butyrates, benzoates, and the like.

By once weekly dosing is meant that a unit dosage of the pharmaceutical active is administered once a week, i.e. one time during a seven day period, preferably on the same day of each week. In the once weekly dosing regimen, the unit dosage is generally administered about once every seven days. A nonlimiting example of a once weekly dosing regimen would entail the administration of a unit dosage of the bisphosphonate every Sunday. Even though not required, it is preferred that the pharmaceutical active be administered on the same day each week in order to maximize the potential for patient compliance and accuracy.

The term “foldable” as used herein means that the main card of the kit of the present invention is capable of being folded or can if desired be folded.

The methods and compositions of the present invention are particularly useful for administering pharmaceutical actives such as bisphosphonates for inhibiting bone resorption and for treating and preventing abnormal bone resorption and conditions associated therewith. Such conditions include both generalized and localized bone loss. Also, the creation of bone having an abnormal structure, as in Paget's disease, can be associated with abnormal bone resorption. The term “generalized bone loss” means bone loss at multiple skeletal sites or throughout the skeletal system. The term “localized bone loss” means bone loss at one or more specific, defined skeletal sites.

Generalized boss loss is often associated with osteoporosis. Osteoporosis is most common in post-menopausal women, wherein estrogen production has been greatly diminished. However, osteoporosis can also be steroid-induced and has been observed in males due to age. Osteoporosis can be induced by disease, e.g. rheumatoid arthritis, it can be induced by secondary causes, e.g., glucocorticoid therapy, or it can come about with no identifiable cause, i.e. idiopathic osteoporosis. Also, osteoarthritis is a condition that is included herein because of its inflammation and associated bone loss. In the present invention, preferred methods include the treatment or prevention of abnormal bone resorption in osteoporotic humans.

Localized bone loss has been associated with periodontal disease, with bone fractures, and with periprosthetic osteolysis (in other words where bone resorption has occured in proximity to a prosthetic implant).

Generalized or localized bone loss can occur from disuse, which is often a problem for those confined to a bed or a wheelchair, or for those who have an immobilized limb set in a cast or in traction.

The methods and compositions of the present invention are useful for treating and or preventing the following conditions or disease states: osteoporosis, which can include post-menopausal osteoporosis, steroid-induced osteoporosis, male osteoporosis, disease-induced osteoporosis, idiopathic osteoporosis; Paget's disease; abnormally increased bone turnover; periodontal disease; localized bone loss associated with periprosthetic osteolysis; bone fractures; osteoarthritis; and rheumatoid arthritis.

### Treatment Kits

The present invention relates to a kit for conveniently and effectively carrying out the methods in accordance with the present invention. Such kits are especially suited for the delivery of solid oral forms such as tablets or capsules. Such a kit preferably includes a number of unit dosages. Such kits can include a card having the dosages oriented in the order of their intended use. An example of such a kit is a “blister pack”. Blister packs are well known in the packaging industry and are widely used for packaging pharmaceutical unit dosage forms. If desired, the blister can be in the form of a childproof blister, i.e. a blister that is difficult for a child to open, yet can be readily opened by an adult. If desired, a memory aid can be provided, for example in the form of numbers, letters, or other markings or with a calendar feature and/or calendar insert, designating the days in the treatment schedule in which the dosages can be administered. Alternatively, placebo dosages, or vitamin or dietary supplements, either in a form similar to or distinct from the pharmaceutical active dosages, can be included.

The treatment kits can be constructed in a variety of forms familiar to one of ordinary skill in the art. The kits comprise at least one unit dosage of a pharmaceutical active for administration according to a once weekly regimen and a means for containing the unit dosages. The kits comprise a means for the weekly administration of a pharmaceutical active. In one embodiment the kits include from about one to about four unit dosages. In another embodiment, the kits include about four unit dosages.

In one embodiment, the means for containing the unit dosages is a card, preferably a card that is capable of being folded. This card will be referred to herein as a main card, or alternatively a principal card or a first card, to distinguish it from additional optional cards, circulars, or other such materials which can be associated with the kit. This main card can be folded with a simple crease, or alternatively, with a double crease, so as to exhibit a spine, similar to the spine of a closed book. The main card can comprise a printable surface, i.e. a surface upon which the product name, appropriate administration instructions, product information, drawings, logos, memory aids, calendar features, etc. can be printed. The main card can comprise a means for containing said unit dosage or dosages and a memory aid for administering said unit dosage or dosages. The main card, especially if it is prepared from two or more laminated paperboard surfaces, can comprise a slit or pocket, preferably in one of the inner paperboard surfaces of the folded card. The slit or pocket can be used to contain a removable secondary card, i.e. a second card or insert card, which is not permanently attached or affixed to the main card.

The memory aid can include a listing of the days of the week, i.e. Sunday, Monday, Tuesday, Wednesday, Thursday, Friday, and Saturday, with appropriate spaces for the patient to select and indicate on the card the preferred day of the week on which to administer the therapy. The memory aid can also include removable stickers having an appropriate pressure sensitive adhesive to facilitate easy removal and refastening to a desired surface such as a calendar or dayminder. The removable stickers can be located on the main card, or can be located on the secondary card which is constructed so that it can be readily inserted into and removed from the optional slit in the main card. Additionally, the optional slit can contain additional patient information and other circulars.

Other means for containing said unit dosages can include bottles and vials, wherein the bottle or vial comprises a memory aid, such as a printed label for administering said unit dosage or dosages. The label can also contain removable reminder stickers for placement on a calendar or dayminder to further help the patient to remember when to take a dosage or when a dosage has been taken.

The Figures exemplify an embodiment of the present invention.

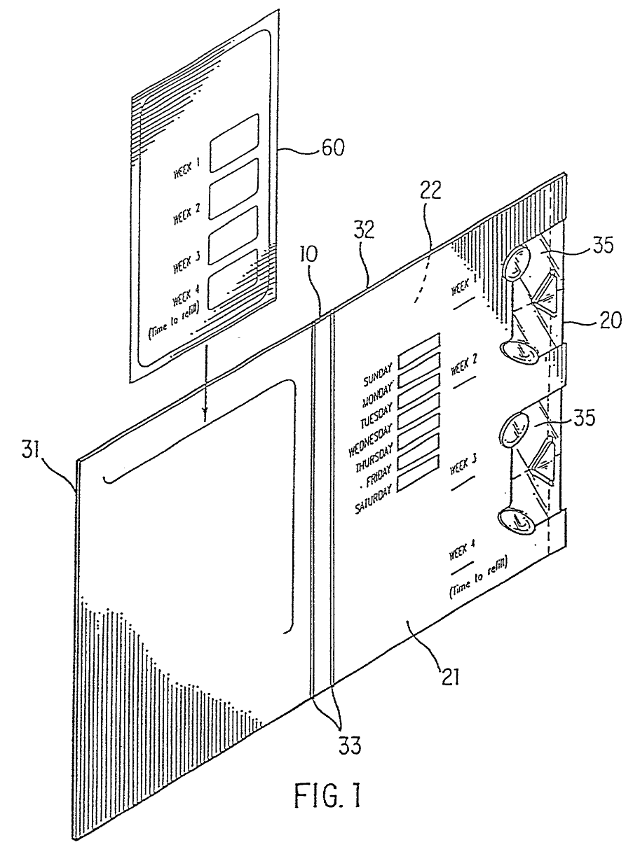

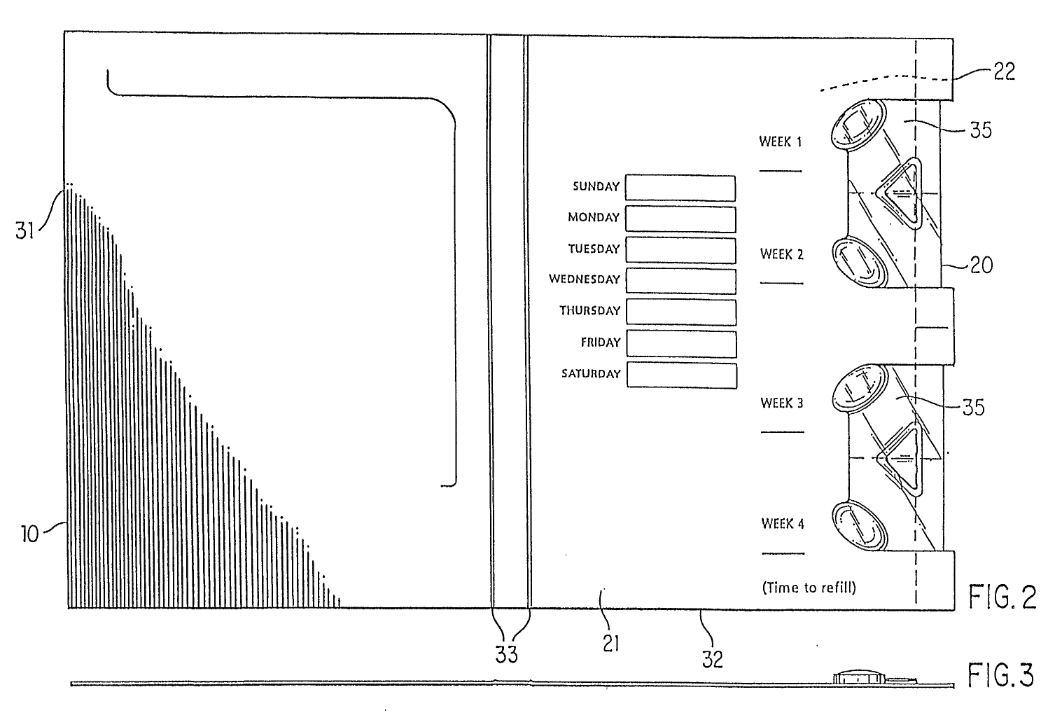

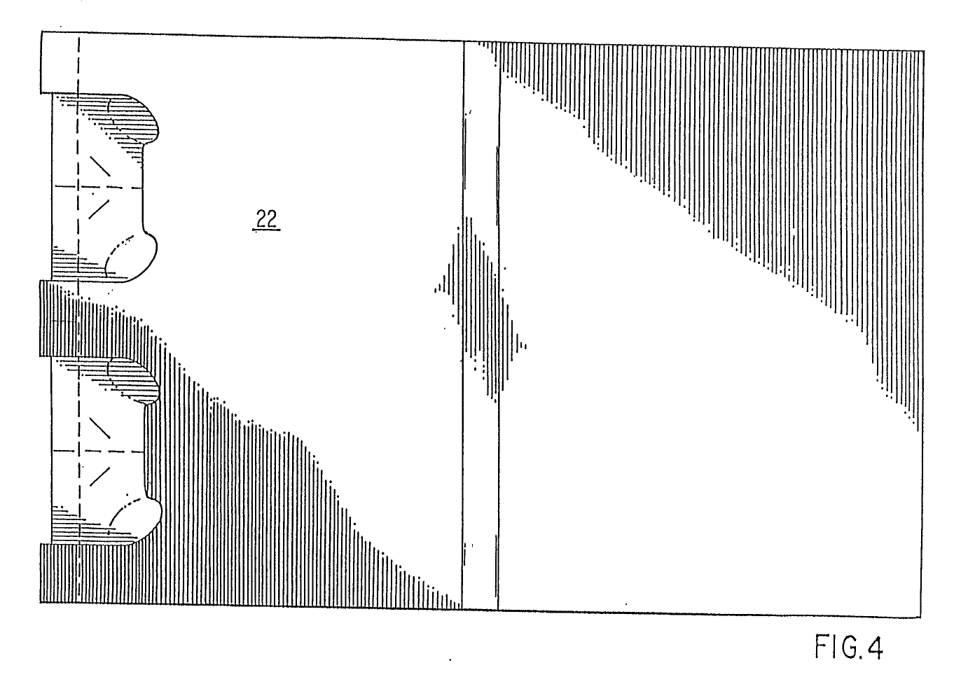

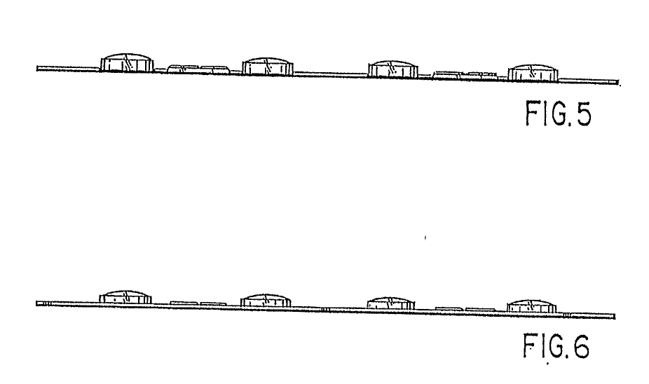

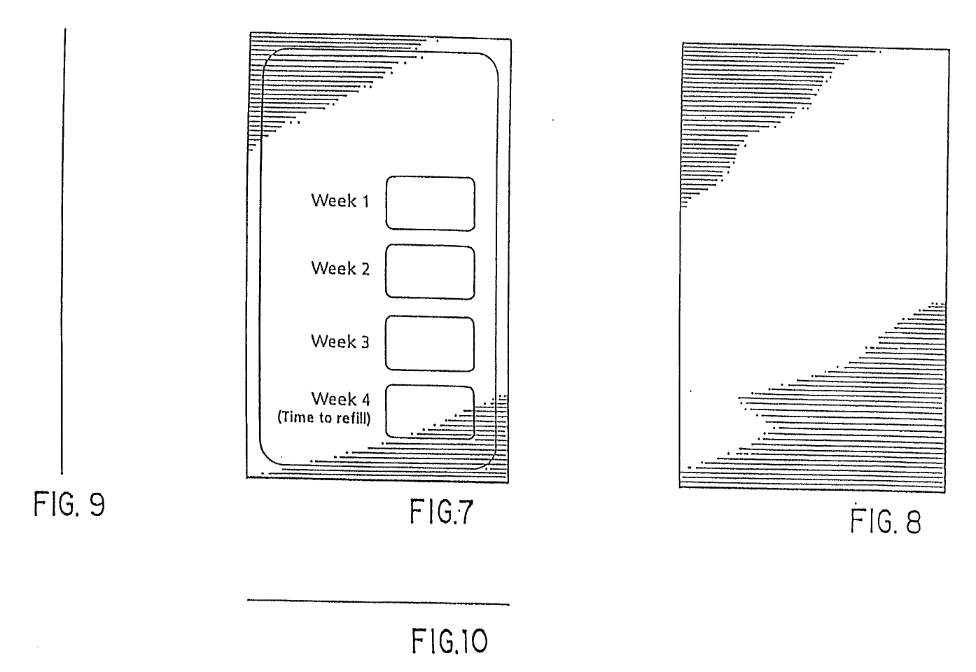

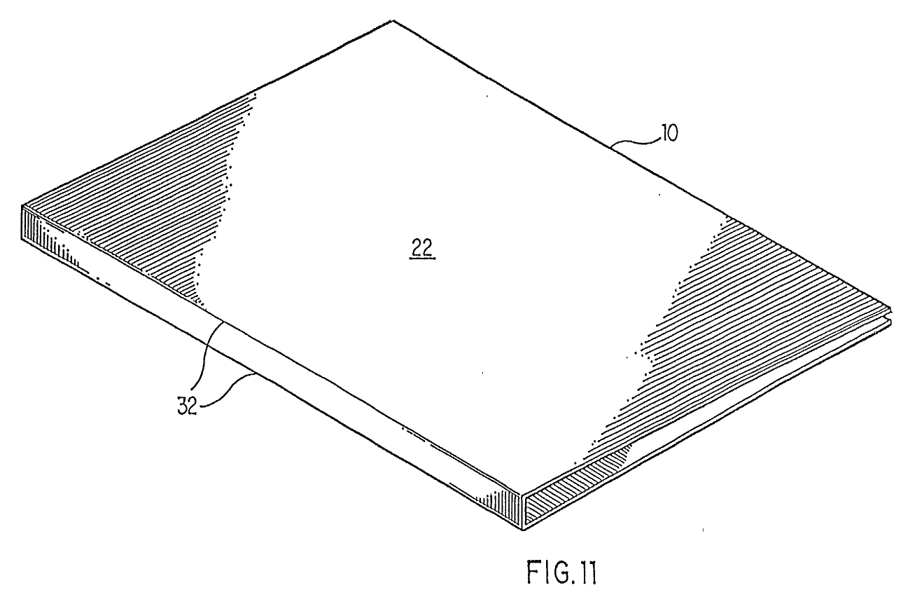

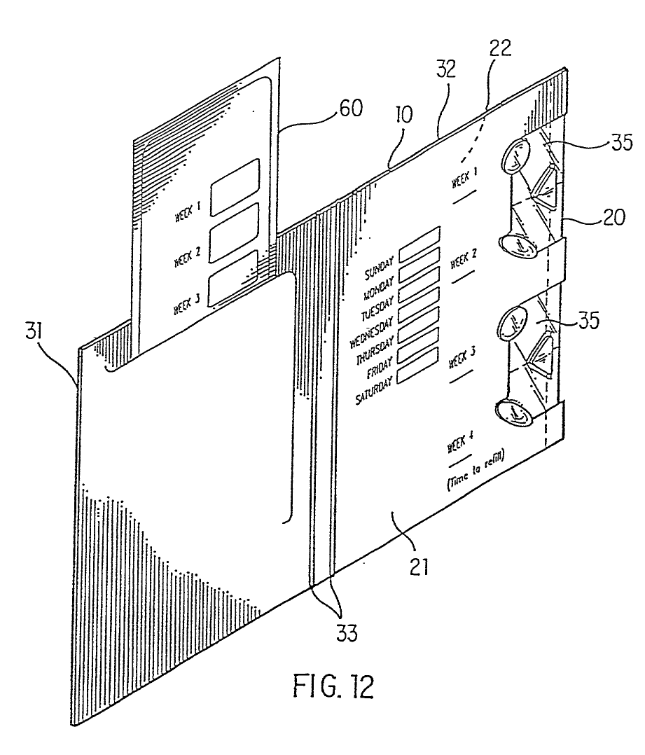

Referring to FIGS. 1, 2, and 12, the main pharmaceutical card (10) is characterized as a uniform edge, multiple layer laminate having proportionally dimension, first (31) and second (32) sides divided by hinge means (33). The first and second sides are characterized by interior (21) and exterior (22) surfaces. On the second side, blister package (20) is affixed between the interior and exterior surfaces. Cut out surface (35) of the blister package exposes the blister and notch cavities. Referring to FIGS. 1 and 12, secondary card (60) is shown as a removable insert.

Referring to FIG. 11 the pharmaceutical treatment card is folded along hinge means (33) to place the card in a closed position, wherein exterior layer (22) of the first side of the card is shown.

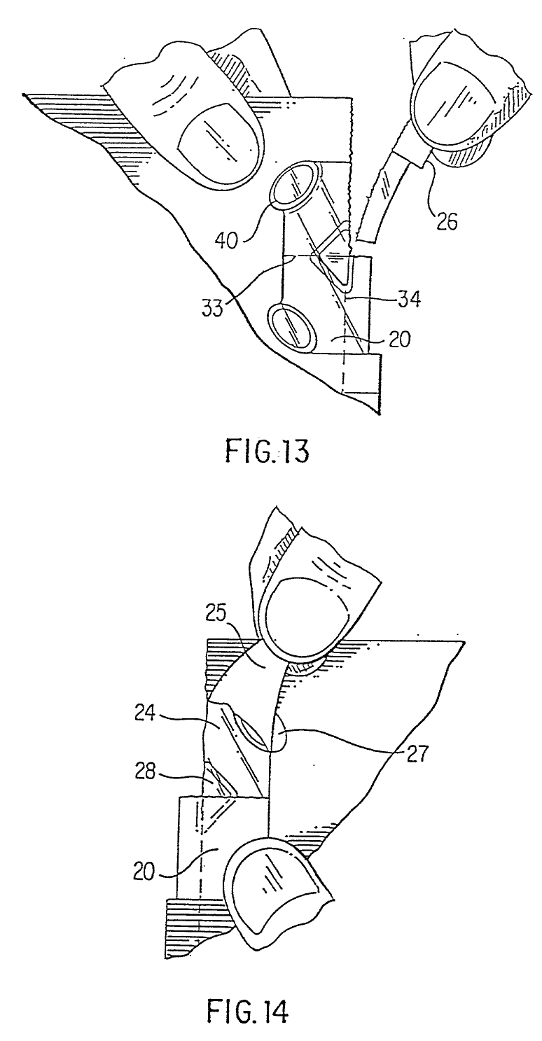

Referring to FIG. 13, barrier strip (26) is being tom from an edge of the card along second, perforation line (34) and a first, perforation line (33) to expose a the multiple layer laminate in the region notch cavity (28). While the card's interior and exterior layers are held between the finger and thumb of a user's first hand in an area of the card proximal to the blister package, the barrier strip can be torn away at the desired individual blister cavity using the second hand.

Referring to FIG. 14, backing layer (25) and a small section of lidding layer (24) are being peeled away from notched cavity (28) towards blister cavity (27) to expose lidding layer (24). While the thumb and fingers of user's first hand grasp the second side of the card, the fore finger and thumb of the user's second hand can grasp a tab comprising an adhered portion of the lidding and backing layers underneath the notch cavity and peels the backing layer away from the notch cavity and towards the blister cavity holding a dosage of the pharmaceutical composition.

Referring to FIG. 15, blister package (20) is shown wherein composition (40) is pushed from the blister cavity (27) through lidding layer (23) to dislodge the composition from an individual blister cavity. While at least one finger and thumb of a user's first hand grasp the card proximal to the desired blister cavity, the thumb of the user's second hand can be utilized to push against the blister cavity, wherein the blister cavity presses against the composition and cause it to contact the lidding layer, rupturing the layer to force the composition therethrough.

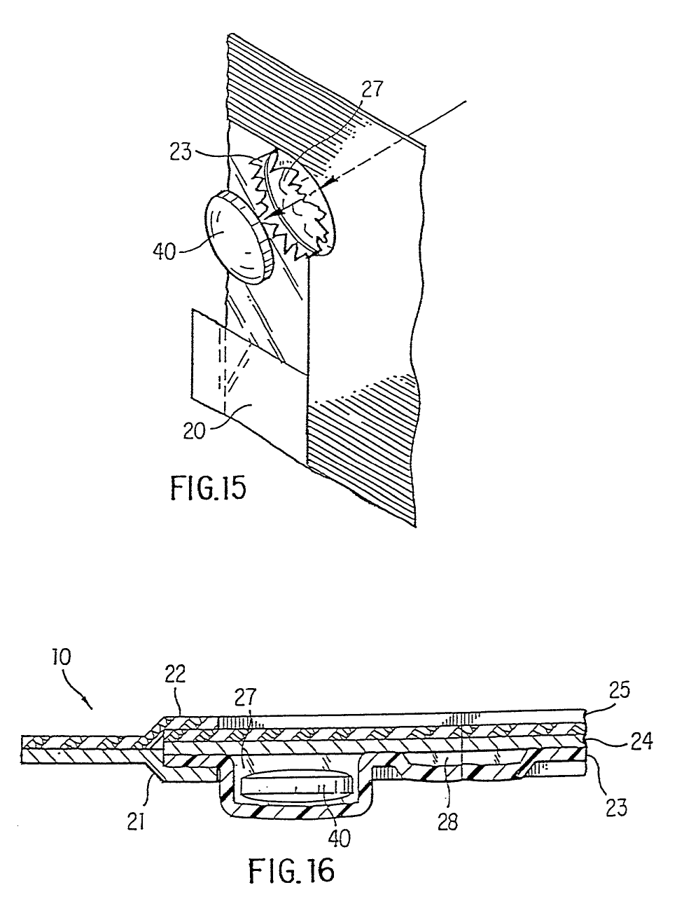

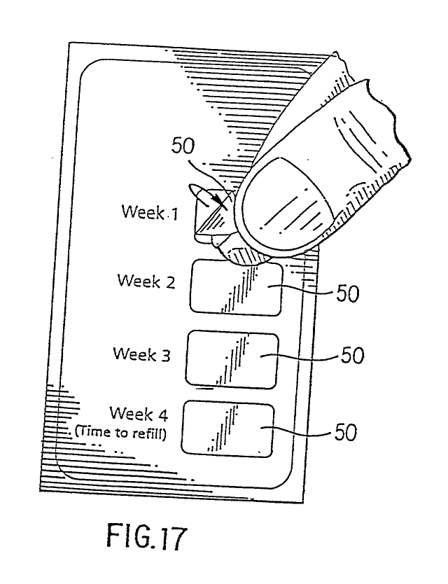

Referring to FIG. 16, blister package (20) is shown in detail as a multiple layer laminated assembly containing an individually, sealed blister cavities (27) containing a pre-measured dosage of a pharmaceutical composition (40). Referring to the layers, the interior layer (21) and exterior layer (22) of the second side of the card provide outer most layers for attachment of the blister package to the card. Immediately adjacent to the interior layer is blister layer (23) characterized as having a plurality of individual blister cavities (27) and a plurality of notch cavities (28) incorporated therein. The interior and blister layers are affixed together by an adhesive. Opposite the interior layer and adjacent to the blister layer is lidding layer (24), wherein a single dosage of pharmaceutical composition (40), located within the blister cavity, is held in place by the lidding layer. The blister and lidding layers are sealed together by an adhesive. Opposite the blister layer and next to the lidding layer is peelable backing layer (25). The lidding and peelable, backing layers are in direct contact with one another without the benefit of an adhesive, except that area of the lidding and backing layers proximal to the notch cavity is attached by an adhesive to form a pull tab. Opposite the lidding layer and next to the peelable backing layer is exterior layer (22) of the card. The peelable backing and exterior layers are held together by an adhesive. The barrier strip (26) is a composite of layers 21, 22, 23, 24 and 25 that have been adhered together (see FIG. 1).

Referring to FIG. 17, a removable sticker (50) is being removed from the secondary card. The removable sticker can be removed using the thumb and forefinger.

The remaining FIGS. Illustrate various components as designated.

Additionally, as used herein the term “uniform edge” is defined as edges of individual layers that are attached together to form a single structure, wherein the edges of several layers in a laminated structure can be affixed together to form one edge.

The term “multiple layer laminate” is defined as a structure having a plurality of separate layers affixed together to form a single layered unit.

The term “interior layer” is defined as the inside surface of the first or second side of a pharmaceutical card that abut one another other when the card is folded along its hinge means. The interior layer can contain indicia in the form of literature and instructional materials, as well as methods of assisting a user to schedule dosage administration intervals.

The term “exterior layer” is defined as the outside surface of a pharmaceutical card, when folded along its hinge means, that can contain indicia in the form of literature and instructional materials, as well as methods of assisting a user to schedule dosage administration intervals.

The term “sealed” as used herein when referring to the blister cavities is defined as an air tight and moisture proof blister cavity having several months shelf life.

As used herein the term “hinge means” means creases, lines or other folding properties incorporated into a pharmaceutical card where the card can be folded along bifurcated sides to define interior and exterior sides thereof.

The term “proximal” is defined as a first component that is about adjacent or sufficiently close to a second component of the card.

The phrase “cut out surface” refers to removed portions of the interior and exterior layers of the second side of the card in the region of the blister package that has been removed to expose the blister and backing layers of said package. In the ‘cut out surface’ the blister and notch cavities as well as the first, perforation lines are visible.

### Pharmaceutical Actives

The kits of the present invention are useful for administering a wide variety of pharmaceutical actives, particularly those that can be administered according to a continuous regimen having a periodicity of about once a week, i.e. once weekly.

Nonlimiting examples of such pharmaceutical actives include: classes of actives such as antibiotics, antifungals, antiinflammatories, hormonal agents, antitumor agents, immunosuppresants, cardiovascular agents, and the like. Particularly useful for such administration are pharmaceutical actives having appropriately long half lives or availability in the body, once administered. Also, various conditions and disease states having target or receptor areas in the body having an intervention period or window during which the pharmaceutical active should be delivered once weekly are well suited for such therapies.

Without being limited by theory, it is believed that bisphosphonate therapy and the inhibition of bone resorption, particularly the treatment and prevention of osteoporosis, is well suited to such once weekly therapy. Many bisphosphonates are believed to actually become incorporated in bone tissue once administered. Once incorporated into bone tissue, the bisphosphonate is believed to have a very long half. Furthermore, it is believed that the bone remodeling units, i.e. the areas in which bone resorption occur throughout the skeleton, have an active period of up to about 21 days. See Fleisch, “Bisphosphonates in Bone Disease From the Laboratory to the Patient”, Third Edition, Parthenon Publishing, ISBN 1-85070-951-3, 1997; Kanis, “Osteoporosis”, Blackwell Science, ISBN 0-632-03811-X, 1994; and Harris, S. T. et al., “The Effect of Short Term Treatment With Alendronate on Vertebral Density and Biochemical Markers of Bone Remodeling in Early Postmenopausal Women”, J. Clin. Endocrinol. Metab., 1993: 76(6), pp. 1399-1406, which are all incorporated by reference herein in their entirety.

In certain embodiments, the methods and compositions of the present invention comprise a bisphosphonate. The bisphosphonates of the present invention correspond to the chemical formula

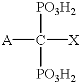

wherein

A and X are independently selected from the group consisting of H, OH, halogen, NH_{2,} SH, phenyl, C1-C30 alkyl, C1-C30 substituted alkyl, C1-C10 alkyl or dialkyl substituted NH_{2,} C1-C10 alkoxy, C1-C10 alkyl or phenyl substituted thio, C1-C10 alkyl substituted phenyl, pyridyl, furanyl, pyrrolidinyl, imidazonyl, and benzyl.

In the foregoing chemical formula, the alkyl groups can be straight, branched, or cyclic, provided sufficient atoms are selected for the chemical formula. The C1-C30 substituted alkyl can include a wide variety of substituents, nonlimiting examples which include those selected from the group consisting of phenyl, pyridyl, furanyl, pyrrolidinyl, imidazonyl, NH_{2,} C1-C10 alkyl or dialkyl substituted NH₂, OH, SH, and C1-C10 alkoxy.

In the foregoing chemical formula, A can include X and X can include A such that the two moieties can form part of the same cyclic structure.

The foregoing chemical formula is also intended to encompass complex carbocyclic, aromatic and hetero atom structures for the A and/or X substituents, nonlimiting examples of which include naphthyl, quinolyl, isoquinolyl, adamantyl, and chlorophenylthio.

Preferred structures are those in which A is selected from the group consisting of H, OH, and halogen, and X is selected from the group consisting of C1-C30 alkyl, C1-C30 substituted alkyl, halogen, and C1-C10 alkyl or phenyl substituted thio.

More preferred structures are those in which A is selected from the group consisting of H, OH, and Cl, and X is selected from the group consisting of C1-C30 alkyl, C1-C30 substituted alkyl, C1, and chlorophenylthio.

Most preferred is when A is OH and X is a 3-aminopropyl moiety, so that the resulting compound is a 4-amino-1,-hydroxybutylidene-1,1-bisphosphonate, i.e. alendronate.

Pharmaceutically acceptable salts and derivatives of the bisphosphonates are also useful herein. Nonlimiting examples of salts include those selected from the group consisting alkali metal, alkaline metal, ammonium, and mono-, di, tri-, or tetra-C1-C30-alkyl-substituted ammonium. Preferred salts are those selected from the group consisting of sodium, potassium, calcium, magnesium, and ammonium salts. Nonlimiting examples of derivatives include those selected from the group consisting of esters, hydrates, and amides. “Pharmaceutically acceptable” as used herein means that the salts and derivatives of the bisphosphonates have the same general pharmacological properties as the free acid form from which they are derived and are acceptable from a toxicity viewpoint.

It should be noted that the terms “bisphosphonate” and “bisphosphonates”, as used herein in referring to the therapeutic agents of the present invention are meant to also encompass diphosphonates, biphosphonic acids, and diphosphonic acids, as well as salts and derivatives of these materials. The use of a specific nomenclature in referring to the bisphosphonate or bisphosphonates is not meant to limit the scope of the present invention, unless specifically indicated. Because of the mixed nomenclature currently in use by those or ordinary skill in the art, reference to a specific weight or percentage of a bisphosphonate compound in the present invention is on an acid active weight basis, unless indicated otherwise herein. For example, the phrase “about 70 mg of a bone resorption inhibiting bisphosphonate selected from the group consisting of alendronate, pharmaceutically acceptable salts thereof, and mixtures thereof, on an alendronic acid active weight basis” means that the amount of the bisphosphonate compound selected is calculated based on 70 mg of alendronic acid.

Nonlimiting examples of bisphosphonates useful herein include the following:

Alendronic acid, 4-amino-1-hydroxybutylidene-1,1-bisphosphonic acid.

Alendronate (also known as alendronate sodium or monosodium trihydrate), 4-amino-1-hydroxybutylidene-1,1-bisphosphonic acid monosodium trihydrate.

Alendronic acid and alendronate are described in U.S. Pat. No. 4,922,007, to Kieczykowski et al., issued May 1, 1990, and U.S. Pat. No. 5,019,651, to Kieczykowski, issued May 28, 1991, both of which are incorporated by reference herein in their entirety. 1,1-dichloromethylene-1,1-diphosphonic acid (clodronic acid), and the disodium salt (clodronate, Procter and Gamble), are described in Belgium Patent 672,205 (1966) and J. Org. Chem 32, 4111 (1967), both of which are incorporated by reference herein in their entirety. 1-hydroxy-3-(1-pyrrolidinyl)-propylidene-1,1-bisphosphonic acid (EB-1053).

1-hydroxyethane-1,1-diphosphonic acid (etidronic acid).

1-hydroxy-3-(N-methyl-N-pentylamino)propylidene-1,1-bisphosphonic acid, also known as BM-210955, Boehringer-Mannheim (ibandronate), is described in U.S. Pat. No. 4,927,814, issued May 22, 1990, which is incorporated by reference herein in its entirety.

Cycloheptylaminomethylene-1,1-bisphosphonic acid, YM 175, Yamanouchi (incadronate, formerly known as cimadronate), as described in U.S. Pat. No. 4,970,335, to Isomura et al., issued Nov. 13, 1990, which is incorporated by reference herein in its entirety.

1-hydroxy-2-imidazo-(1,2-a)pyridin-3-yethylidene (minodronate).

6-amino-1-hydroxyhexylidene-1,1-bisphosphonic acid (neridronate).

3-(dimethylamino)-1-hydroxypropylidene-1,1-bisphosphonic acid (olpadronate).

3-amino-1-hydroxypropylidene-1,1-bisphosphonic acid (pamidronate).

[2-(2-pyridinyl)ethylidene]-1,1-bisphosphonic acid (piridronate) is described in U.S. Pat. No. 4,761,406, which is incorporated by reference in its entirety.

1-hydroxy-2-(3-pyridinyl)-ethylidene-1,1-bisphosphonic acid (risedronate).

(4-chlorophenyl)thiomethane-1,1-disphosphonic acid (tiludronate) as described in U.S. Pat. No. 4,876,248, to Breliere et al., Oct. 24, 1989, which is incorporated by reference herein in its entirety.

1-hydroxy-2-(1H-imidazol-1-yl)ethylidene-1,1-bisphosphonic acid (zolendronate).

Preferred are bisphosphonates selected from the group consisting of alendronate, clodronate, etidronate, ibandronate, incadronate, minodronate, neridronate, risedronate, piridronate, pamidronate, tiludronate, zoledronate, pharmaceutically acceptable salts or esters thereof, and mixtures thereof.

More preferred is alendronate, ibandronate, risedronate, pharmaceutically acceptable salts or esters thereof, and mixtures thereof.

More preferred is alendronate, pharmaceutically acceptable salts thereof, and mixtures thereof.

Most preferred is alendronate monosodium trihydrate.

In other embodiments, other preferred salts are the sodium salt of ibandronate, and risedronate monosodium hemi-pentahydrate (i.e. the 2.5 hydrate of the monosodium salt).

The dosage regimen utilizing the compounds of the present invention is selected in accordance with a variety of factors including type, species, age, weight, sex and medical condition of the patient or subject; the severity of the condition to be treated; the route of administration; the renal and hepatic function of the patient or subject; and the particular compound or salt thereof employed. An ordinarily skilled physician, veterinarian or clinician can readily determine and prescribe the effective amount of the drug required to treat or reduce the risk of contracting the condition.

Compositions useful in the present invention comprise a pharmaceutically effective amount of a bisphosphonate. The bisphosphonate is typically administered in admixture with suitable pharmaceutical diluents, excipients, or carriers, collectively referred to herein as “carrier materials”, suitably selected with respect to oral administration, i.e. tablets, capsules, elixirs, syrups, effervescent compositions, powders, and the like, and consistent with conventional pharmaceutical practices. For example, for oral administration in the form of a tablet, capsule, or powder, the active ingredient can be combined with an oral, non-toxic, pharmaceutically acceptable inert carrier such as lactose, starch, sucrose, glucose, methyl cellulose, magnesium stearate, mannitol, sorbitol, croscarmellose sodium and the like; for oral administration in liquid form, e.g., elixirs and syrups, effervescent compositions, the oral drug components can be combined with any oral, non-toxic, pharmaceutically acceptable inert carrier such as ethanol, glycerol, water and the like. Moreover, when desired or necessary, suitable binders, lubricants, disintegrating agents, buffers, coatings, and coloring agents can also be incorporated. Suitable binders can include starch, gelatin, natural sugars such a glucose, anhydrous lactose, free-flow lactose, beta-lactose, and corn sweeteners, natural and synthetic gums, such as acacia, guar, tragacanth or sodium alginate, carboxymethyl cellulose, polyethylene glycol, waxes, and the like. Lubricants used in these dosage forms include sodium oleate, sodium stearate, magnesium stearate, sodium benzoate, sodium acetate, sodium chloride and the like. A particularly preferred tablet formulation for alendronate monosodium trihydrate is that described in U.S. Pat. No. 5,358,941, to Bechard et al, issued Oct. 25, 1994, which is incorporated by reference herein in its entirety. The compounds used in the present method can also be coupled with soluble polymers as targetable drug carriers. Such polymers can include polyvinylpyrrolidone, pyran copolymer, polyhydroxylpropyl-methacrylamide, and the like.

The precise dosage of the bisphonate will vary with the dosing schedule, the oral potency of the particular bisphosphonate chosen, the age, size, sex and condition of the mammal or human, the nature and severity of the disorder to be treated, and other relevant medical and physical factors. Thus, a precise pharmaceutically effective amount cannot be specified in advance and can be readily determined by the caregiver or clinician. Appropriate amounts can be determined by routine experimentation from animal models and human clinical studies. Generally, an appropriate amount of bisphosphonate is chosen to obtain a bone resorption inhibiting effect, i.e. a bone resorption inhibiting amount of the bisphosphonate is administered. For humans, an effective oral dose of bisphosphonate is typically from about 1.5 to about 6000 μg/kg body weight and preferably about 10 to about 2000 μg/kg of body weight.

For human oral compositions comprising alendronate, pharmaceutically acceptable salts thereof, or pharmaceutically acceptable derivatives thereof, a unit dosage typically comprises from about 8.75 mg to about 140 mg of the alendronate compound, on an alendronic acid active weight basis, i.e. on the basis of the corresponding acid.

For human oral compositions comprising ibandronate, pharmaceutically acceptable salts thereof, or pharmaceutically acceptable derivatives thereof, a unit dosage typically comprises from about 3.5 mg to about 200 mg of the ibandronate compound, on an ibandronic acid active weight basis, i.e. on the basis of the corresponding acid.

For human oral compositions comprising risedronate, pharmaceutically acceptable salts thereof, or pharmaceutically acceptable derivatives thereof, a unit dosage typically comprises from about 3.5 mg to about 200 mg of the risedronate compound, on a risedronic acid active weight basis, i.e. on the basis of the corresponding acid.

For once weekly dosing, an oral unit dosage comprises from about 17.5 mg to about 70 mg of the alendronate compound, on an alendronic acid active weight basis. Examples of weekly oral dosages include a unit dosage which is useful for osteoporosis prevention comprising about 35 mg of the alendronate compound, and a unit dosage which is useful for treating osteoporosis comprising about 70 mg of the alendronate compound.

For once weekly dosing, an oral unit dosage comprises from about 3.5 mg to about 200 mg, preferably from about 7 mg to about 100 mg of the ibandronate compound, on an ibandronic acid active weight basis, i.e. calculated on the basis of the corresponding acid. Examples of weekly oral dosages include a unit dosage which is useful for inhibiting bone resorption, and treating and preventing osteoporosis selected from the group consisting of about 25 mg, 30 mg, 35 mg, 40 mg, 45 mg, or 50 mg.

For once weekly dosing, an oral unit dosage comprises from about 3.5 mg to about 200 mg, preferably from about 7 mg to about 100 mg of the risedronate compound, on a risedronic acid active weight basis, i.e. calculated on the basis of the corresponding acid. Examples of weekly oral dosages include a unit dosage which is useful for inhibiting bone resorption, and treating and preventing osteoporosis selected from the group consisting of about 25 mg, 30 mg, 35 mg, 40 mg, 45 mg, or 50 mg.

Nonlimiting examples of oral compositions comprising alendronate, as well as other bisphosphonates, are illustrated in the Examples, below.

### EXAMPLES

The following examples further describe and demonstrate embodiments within the scope of the present invention. The examples are given solely for the purpose of illustration and are not to be construed as limitations of the present invention as many variations thereof are possible without departing from the spirit and scope of the invention.

### Example 1

A kit for administering a pharmaceutical active is made up as follows:

A paperboard laminate card, i.e. a first card, of two paperboard components with a CR blister containing the desired number of blisters (e.g., about 4 blisters) sealed between the laminates layers is constructed. Each paperboard component has a thickness of about 0.010 inches (about 0.0035 cm) such that the double laminate has a total thickness of about 0.020 inches (about 0.0070 cm). The overall dimension of the double laminated card is about 5½ inches (about 13.97 cm) by about 8 5/16 inches (about 21.11 cm). The card is folded in half with a double crease to form a spine of about 5 5/16 inches (about 0.79 cm) such that the folded card resembles a book having the dimensions of about 5½ inches (about 13.97 cm) by about 4 inches (about 10.16 cm). The spine helps ensure that the card folds appropriately over the blisters. In other embodiments, the card is folded with a single crease such that the folded card has dimensions of about 5 ½ inches (about 13.97 cm) by about 4 3/32 inches (about 10.555 cm). The card is optionally constructed to contain a slit for further containing a flat paperboard card, i.e. a second card. This second card can be further a fixed with reminder stickers having an appropriate pressure sensitive adhesive for allowing easy removal and fixation to another surface, such as a calendar or daily reminder. The paperboard double laminated card is imprinted to indicate the days of the week, i.e. Sunday, Monday, Tuesday, Wednesday, Thursday, Friday, and Saturday, so that a patient can select a preferred day for administering the pharmaceutical active. Each blister(s) of the card contain(s) a unit dosage of the pharmaceutical active to be administered.

### Example 2

Once weekly dosing regimen.

Treatment of osteoporosis.

Alendronate tablet formulations containing about 70 mg of alendronate, on an alendronic acid active basis, are prepared (see EXAMPLE 5). The tablets are orally administered to a human patient once weekly, i.e. preferably about once every seven days (for example, every Sunday), for a period of at least one year from the pharmaceutical kit described above in Example 1. A card containing four tablets provides a convenient four week course of therapy with additional kits being obtained as needed. This method of administration is useful and convenient for treating osteoporosis. This method is also useful for improving patient acceptance and compliance.

### Example 3

Once weekly dosing regimen

Prevention of osteoporosis.

Alendronate tablet formulations containing about 35 mg of alendronate, on an alendronic acid active basis, are prepared (see EXAMPLE 5). The tablets are orally administered to a human patient once weekly, i.e. preferably about once every seven days (for example, every Sunday), for a period of at least one year from the pharmaceutical kit described above in Example 1. A card containing four tablets provides a convenient four week course of therapy with additional kits being obtained as needed. This method of administration is useful and convenient for preventing osteoporosis. This method is also useful for improving patient acceptance and compliance.

### Example 4

In further embodiments, alendronate tablets are orally dosed, at the desired dosage, according to the weekly dosing schedule for treating or preventing other disorders associated with abnormal bone resorption.

### Example 5

Bisphosphonate tablets.

Bisphosphonate containing tablets are prepared using standard mixing and formation techniques as described in U.S. Pat. No. 5,358,941, to Bechard et al., issued Oct. 25, 1994, which is incorporated by reference herein in its entirety.

Tablets containing about 35 mg of alendronate, on an alendronic acid active basis, are prepared using the following relative weights of ingredients.

| Ingredient | Per Tablet | Per 4000 Tablets |
|---|---|---|
| Alendronate Monosodium Trihydrate | 45.68 mg | 182.72 g |
| Anhydrous Lactose, NF | 71.32 mg | 285.28 g |
| Microcrystalline Cellulose, NF | 80.0 mg | 320.0 g |
| Magnesium Stearate, NF | 1.0 mg | 4.0 g |
| Croscarmellose Sodium, NF | 2.0 mg | 8.0 g |

The resulting tablets are useful for administration in accordance with the methods of the present invention for inhibiting bone resorption.

Similarly, tablets comprising other relative weights of alendronate, on an alendronic acid active basis are prepared: e.g., about 8.75, 17.5, 70, and 140 mg per tablet. Also, tablets containing other bisphosphonates at appropriate active levels are similarly prepared: e.g., clodronate, etidronate, ibandronate, incadronate, minodronate, neridronate, risedronate, piridronate, pamidronate, tiludronate, zoledronate, pharmaceutically acceptable salts or esters thereof, and mixtures thereof. Also, tablets containing combinations of bisphosphonates are similarly prepared.

Examples of such tablets include tablets containing about 25 mg, 30 mg, 35 mg, 40 mg, 45 mg, or 50 mg of a risedronate compound, particularly risedronate monosodium hemi-pentahydrate, on an acid, i.e. risedronic acid, active basis. Other Examples of such tablets include tablets containing about 25 mg, 30 mg, 35 mg, 40 mg, 45 mg, or 50 mg of an ibandronate compound on an acid, i.e. ibandronic acid, active basis.

What is claimed is:

1. A kit for administering a pharmaceutical active according to a once weekly regimen, said kit comprising:
(a) at least one unit dosage of a pharmaceutical active for administration according to a once weekly regimen, and
(b) a card, said card further comprising:
(i) at least one printable surface,
(ii) a means for containing said unit dosage or dosages, and
(iii) a memory aid for administering said unit dosage or dosages.

2. A kit according to claim 1 wherein said card is foldable.

3. A kit according to claim 2 wherein said means for containing said unit dosage or dosages is a blister.

4. A kit according to claim 3 wherein said unit dosages are oriented in the intended order of their use.

5. A kit according to claim 4 wherein said card is paperboard.

6. A kit according to claim 5 wherein said memory aid comprises a calendar for indicating the day of the week on which to administer said dosage or dosages.

7. A kit according to claim 6 wherein said memory aid comprises a printable removable sticker corresponding to said dosage or dosages for indicating the administration of said dosage or dosages.

8. A kit according to claim 5 wherein said card further comprises a slit.

9. A kit according to claim 8 which further comprises a removable unfolded card which is insertable in said slit.

10. A kit according to claim 9 wherein said memory aid comprises a printable removable sticker corresponding to said dosage or dosages for indicating the administration of said dosage or dosages.

11. A kit according to any of claims 1-10 wherein said pharmaceutical active is a bisphosphonate.

12. A kit according to claim 11 wherein said bisphosphonate is selected from the group consisting of alendronate, clodronate, etidronate, ibandronate, incadronate, minodronate, neridronate, risedronate, piridronate, pamidronate, tiludronate, zoledronate, pharmaceutically acceptable salts or esters thereof, and mixtures thereof.

13. A kit according to claim 12 wherein said bisphosphonate is selected from the group consisting of alendronate, pharmaceutically acceptable salts or esters thereof, and mixtures thereof.

14. A kit according to claim 13 wherein said unit dosage comprises from about 8.75 mg to about 140 mg of alendronate on an alendronic acid active basis.

15. A kit according to claim 14 wherein said pharmaceutically acceptable salt is alendronate monosodium trihydrate.

16. A kit according to claim 14 wherein said unit dosage comprises about 35 mg of alendronate on an acid active basis.

17. A kit according to claim 16 wherein said pharmaceutically acceptable salt is alendronate monosodium trihydrate.

18. A kit according to claim 14 wherein said unit dosage comprises about 70 mg of alendronate on an acid active basis

19. A kit according to claim 18 wherein said pharmaceutically acceptable salt is alendronate monosodium trihydrate.

20. A kit according to claim 11 wherein said bisphosphonate is selected from the group consisting of risedronate, pharmaceutically acceptable salts or esters thereof, and mixtures thereof.

21. A kit according to claim 20 wherein said unit dosage comprises from about 3.5 mg to about 200 mg of risedronate on a risedronic acid active basis.

22. A kit according to claim 21 wherein said pharmaceutically acceptable salt is risedronate monosodium hemi-pentahydrate.

23. A kit according to claim 20 wherein said unit dosage comprises about 30 mg of risedronate on a risedronic acid active basis.

24. A kit according to claim 23 wherein said pharmaceutically acceptable salt is risedronate monosodium hemi-pentahydrate.

25. A kit according to claim 20 wherein said unit dosage comprises about 35 mg of risedronate on a risedronic acid active basis.

26. A kit according to claim 25 wherein said pharmaceutically acceptable salt is risedronate monosodium hemi-pentahydrate.

27. A kit according to claim 20 wherein said unit dosage comprises about 40 mg of risedronate on a risedronic acid active basis.

28. A kit according to claim 27 wherein said pharmaceutically acceptable salt is risedronate monosodium hemi-pentahydrate.

29. A kit according to claim 20 wherein said unit dosage comprises about 45 mg of risedronate on a risedronic acid active basis.

30. A kit according to claim 29 wherein said pharmaceutically acceptable salt is risedronate monosodium hemi-pentahydrate.

31. A kit according to claim 20 wherein said unit dosage comprises about 50 mg of risedronate on a risedronic acid active basis.

32. A kit according to claim 31 wherein said pharmaceutically acceptable salt is risedronate monosodium hemi-pentahydrate.

33. A kit according to claim 11 wherein said bisphosphonate is selected from the group consisting of ibandronate, pharmaceutically acceptable salts or esters thereof, and mixtures thereof.

34. A kit according to claim 33 wherein said unit dosage comprises from about 3.5 mg to about 200 mg of ibandronate on an ibandronic acid active basis.

35. A kit according to claim 34 wherein said unit dosage comprises about 30 mg of ibandron ate on an ibandronic acid active basis.

36. A kit according to claim 34 wherein said unit dosage comprises about 35 mg of ibandronate on an ibandronic acid active basis.

37. A kit according to claim 34 wherein said unit dosage comprises about 40 mg of ibandronate on an ibandronic acid active basis.

38. A kit according to claim 34 wherein said unit dosage comprises about 45 mg of ibandronate on an ibandronic acid active basis.

39. A kit according to claim 34 wherein said unit dosage comprises about 50 mg of ibandronate on an ibandronic acid active basis.

40. A method for administering a pharmaceutical active according to a continuous schedule having a dosing interval of once weekly comprising administering said pharmaceutical active from a kit comprising
(a) at least one unit dosage of a pharmaceutical active for administration according to a once weekly regimen, and
(b) a card, said card further comprising:
(i) at least one printable surface,
(ii) a means for containing said unit dosage or dosages, and
(iii) a memory aid for administering said unit dosage or dosages.

41. A method according to claim 40 wherein said pharmaceutical active is a bisphosphonate is selected from the group consisting of alendronate, clodronate, etidronate, ibandronate, incadronate, minodronate, neridronate, risedronate, piridronate, pamidronate, tiludronate, zoledronate, pharmaceutically acceptable salts or esters thereof, and mixtures thereof.

42. A method for inhibiting bone resorption in a mammal in need thereof comprising orally administering to said mammal a pharmaceutically effective amount of a bisphosphonate as a unit dosage according to continuous schedule having a dosing interval of once weekly comprising administering said pharmaceutical active from a kit comprising
(a) at least one unit dosage of a pharmaceutical active for administration according to a once weekly regimen, and
(b) a card, said card further comprising:
(i) at least one printable surface,
(ii) a means for containing said unit dosage or dosages, and
(iii) a memory aid for administering said unit dosage or dosages.

43. A method according to claim 42 wherein said bisphosphonate is selected from the group consisting of alendronate, clodronate, etidronate, ibandronate, incadronate, minodronate, neridronate, risedronate, piridronate, pamidronate, tiludronate, zoledronate, pharmaceutically acceptable salts or esters thereof, and mixtures thereof.

44. A method according to claim 43 for treating osteoporosis.

45. A method according to claim 44 for preventing osteoporosis.
